(19) United States
(12) Patent Application Publication
(10) Pub. No.: US 2005/0033403 A1
(43) Pub. Date: Feb. 10, 2005

(54) Implant delivery device

(75) Inventors: John H. Ward, Bloomington, IN (US); Marvin O. Andrews, Bloomington, IN (US); Frank J. Fischer JR., Bloomington, IN (US)

Correspondence Address: BRINKS HOFER GILSON & LIONE, P.O. BOX 10395, CHICAGO, IL, 60610, US

(73) Assignee: Vance Products, Inc. d/b/a Cook Urological Incorporated

(21) Appl. No.: 10/899,807

(22) Filed: Jul. 27, 2004

Related U.S. Application Data
(60) Provisional application No. 60/491,781, filed on Aug. 1, 2003.

(51) Int. Cl. A61F 2/06

(52) U.S. Cl. 623/1.11

(57) ABSTRACT

A delivery device for placing an implant or prosthesis inside a body is described. The device is useful for placing a variety of stents or implants when used with a cystoscope or other visualization or placement device. An inner sheath is relatively stiff and an outer sheath is relatively pliable or soft compared to the inner sheath. The sheaths are assembled to a connector and a positioner is then inserted through the center of the assembly, the inner sheath. The implant is placed near the distal end of the outer sheath, and the assembled device with the implant is backloaded onto a cystoscope or endoscope for placement. The inner sheath may be used to tailor the stiffness or rigidity of the assembled delivery device.

This application claims the benefit of the filing date under 35 U.S.C. § 119(e) of Provisional U.S. Patent Application Ser. No. 60/491,781, filed on Aug. 1, 2003, which is hereby incorporated by reference in its entirety.

### FIELD OF THE INVENTION

The technical field is that of medical devices, and in particular a device for delivering an implant, such as a stent, inside a body of a patient.

### BACKGROUND

A large number of devices have been invented and devised for implanting into the body. These devices may include stents, such as prostatic stents, meant for placement into a male to replace a portion of the urethra that is removed during a radical prostatectomy. Other implants or prostheses may also be implanted to obtain a patent lumen. Other applications may include temporarily replacing or protecting a trachea, a ureter or a bile duct. Other stents may be used for temporary or permanent placement in the vascular system, such as stents to correct stenoses or aneurysms. In addition, tissue or other implants may be used to repair damaged, ulcerated or herniated tissues. For instance, a lumen may be needed after prostate treatment with thermal or chemical ablation, or after radio-frequency treatment.

A central problem in the use of these implants is the safe delivery of the implant to the desired site with minimal trauma to the repair area and to the path used to deliver the implant. The instruments used must be as small, narrow and atraumatic as possible. In addition, the surgeon must typically be able to visualize the operating field, so that he or she can deliver the implant to the precise position in which installation is desired. Finally, the implant delivery device must have sufficient flexibility to traverse bodily passages, and yet must have sufficient rigidity to be able to maneuver the implant or stent to the desired position.

A number of placement devices have been invented to assist in the placement of these stents, but there are disadvantages with their use. For instance, U.S. Pat. No. 5,601,591 discloses a placement device for a prostatic stent. The placement device comprises a stainless steel outer sheath with a blunt expanded distal end. The expansion at the distal end may cause discomfort as it traverses the urethra on its way to replace the prostatic urethral portion. In addition, this placement device has a flexible portion at the proximal end, not the distal end. This will not allow for flexibility where it is most needed, at the point of delivery of the urethral stent.

U.S. Pat. No. 5,593,412 provides a placement device with an outer sheath. The sheath material is described as a polymeric plastic material that softens when a warm saline solution is circulated within the device. After the sheath has been softened, it is more easily retracted, leaving the stent in place. This device requires a continuous source of warm, circulating water or other fluid. This requirement may undesirably add to the diameter of the device; requiring circulating water is undesirable in that this is one more utility in an-already crowded operating area.

U.S. Pat. No. 5,725,571 reveals another implant delivery device, including an outer sheath and an inner device that releasably grips the implant. This device has no taper at the distal end, and may cause trauma on its path to the desired delivery site. U.S. Pat. No. 6,342,066 discloses a delivery device that may be flexible or stiff at its distal end, but has a very complicated structure and also may have an extra sheath at its distal end that undesirably adds to the diameter that must traverse a bodily vessel in order to arrive at the delivery site.

The present invention is aimed at overcoming these deficiencies and providing an implant delivery device that is as thin as possible, atraumatic, and effective in delivering a stent or prosthesis to the precise location desired.

### BRIEF SUMMARY

There are many aspects of the present invention, and many ways to use the invention. One aspect of the invention is a positioning device, comprising an outer sheath and an inner sheath within the outer sheath. The positioning device also comprises a positioner comprising a proximal portion and a flexible distal portion, and a connector, wherein the outer sheath mounts to the connector and the positioner mounts to an inside of the connector.

Another aspect of the invention is a positioning device, the positioning device comprising a tapered outer sheath, an inner sheath within the outer sheath, and a positioner having a proximal end and a distal end. The positioning device also includes a connector, wherein the outer sheath mounts to an outside of the connector and the positioner mounts to an inside of the connector.

Another aspect of the invention is a device for positioning a urethral stent. The device comprises a pliable tapered outer sheath further comprising at least one slit, and a stiff inner sheath within the outer sheath. The device also comprises a positioner having a proximal end and a flexible distal end, and a connector, wherein the outer sheath and inner sheath mount fixedly to the connector.

Yet another aspect of the invention is a method of making a positioning device. The method comprises assembling an inner sheath to a connector, assembling an outer sheath to the connector, and loading a positioner through the connector and the sheaths, the positioner further comprising a flexible distal portion.

Another aspect of the invention is a method of delivering an implant. The method comprises providing an implant delivery device further comprising a sheath with at least one slit, and loading an implant into the delivery device, and also loading a positioner with a flexible distal portion adjacent the implant. The method then comprises determining a location for the implant with a therapeutic or diagnostic medical device, and delivering the implant.

Other systems, methods, features, and advantages of the invention will be or will become apparent to one skilled in the art upon examination of the following figures and detailed description. All such additional systems, methods, features, and advantages are intended to be included within this description, within the scope of the invention, and protected by the accompanying claims.

### BRIEF DESCRIPTION OF THE DRAWINGS

FIG. 1 is an exploded view of a portion of the placement device;

FIG. 2 is a more detailed view of the connector of FIG. 1;

FIGS. 3a and 3b are plan views of a positioner;

FIG. 4 is a perspective view of an alternative embodiment of an outer sheath;

FIG. 5 is a cross sectional view of an assembly of an embodiment together with a cystoscope and a stent for insertion of the stent into a patient; and

FIG. 6 is a flowchart for a process of assembling an implant delivery device, including a stent for delivery.

FIG. 7 is a partial cross-sectional view of an implant delivery device and a handle for operating the device.

FIGS. 8 and 9 are plan views of components useful in embodiments of the delivery device of FIG. 7.

FIG. 10 is a flowchart for a method of delivering an implant.

### DETAILED DESCRIPTION OF THE DRAWINGS AND THE PRESENTLY PREFERRED EMBODIMENTS

Embodiments of the present invention may be used for placement or delivery of a number of different types of devices into the human body. These devices will be most useful in placing stents, for instance. Stents may include prostatic stents, which are used in the urethra of a male after a radical prostatectomy. They may include ureteral stents, placed between a kidney and a bladder to allow flow of urine when flow through the ureter is blocked. The device may also be used for implanting vascular stents and for tissue repair during body healing. The placement devices described herein may be used for placing many of these devices with less trauma to tissue and less discomfort to the patient.

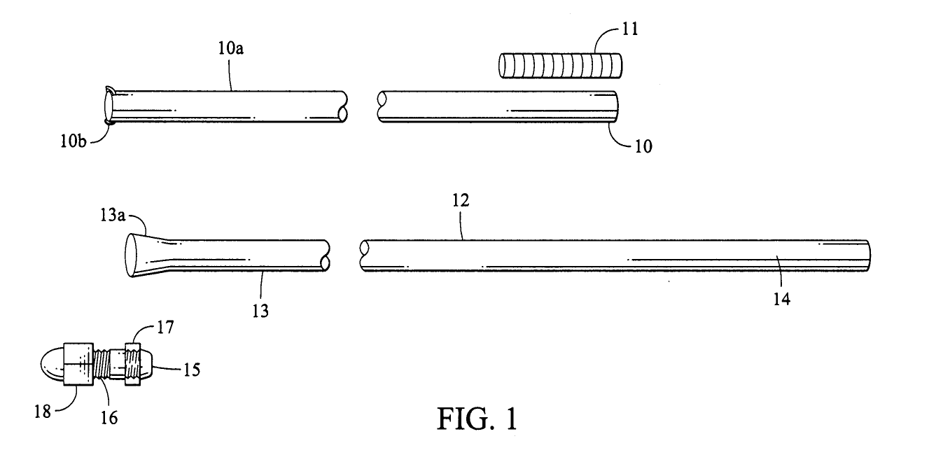

FIG. 1 presents an exploded view of portions of a first embodiment of a delivery device for delivering a prostatic stent. There is an absorbable prostatic stent 11 and an inner sheath 10. There is an outer sheath 12, the outer sheath including a proximal portion 13 and a distal portion 14. Distal portion 14 may have a slight taper toward the distal end for holding stent 11. The outer sheath may instead be designed for a very slight interference fit with the implant to be delivered, in order to retain the implant. Connector 15 has a threaded body 16 and also includes a first nut 17 for threading onto a distal end of the connector, and a second nut 18 for threading onto the proximal end of the connector.

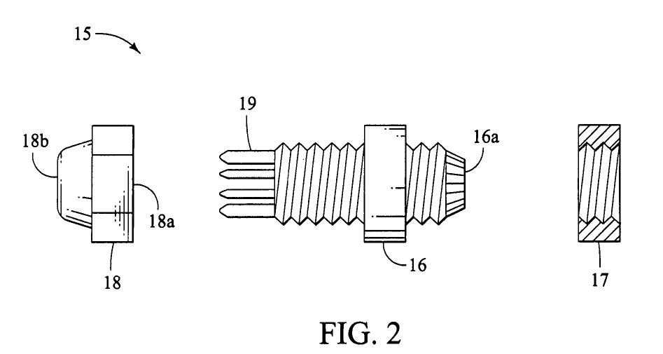

A closer, exploded view of connector 15 is seen in FIG. 2. Connector 15 includes threaded body 16 with a lumen 16a therethrough. First nut 17 threads onto a first or distal end of threaded body 16. Second nut 18 with a lumen 18a therethrough also threads onto threaded body 16, at a proximal end thereof. Nut 18 may also have a domed portion 18b wherein the radius of the dome decreases in the proximal direction. The decrease in radius of the dome causes fingers 19 on threaded body 16 to move radially inwardly when nut 18 is tightened onto threaded body 16.

These component parts of a delivery device work in the following manner. A proximal portion 10a of inner sheath 10 is fixedly mounted inside the distal portion of connector 15. Inner sheath 10 may be adhered to connector 15 with an adhesive or may be fixedly mounted by tabs 10b on the sheath that fit into slots (not shown) on the inner surface of connector 15. Thus, inner sheath 10 is fixedly mounted to connector 15. Outer sheath 12 may also be fixedly mounted to connector 15 by pulling outer sheath 12 and its proximal portion 13 with tapered portion 13a over threads of the distal portion of threaded body 16, and then engaging the threads with nut 17. This also locks the outer sheath into position with respect to both inner sheath 10 and connector 15.

In other embodiments, inner sheath 10 may include tabs 10b that merely slide over a retainer or retaining ring on an inner surface of the distal end of connector 15. In some embodiments, it is important that inner sheath 10 is fixed axially with respect to connector 15. In other embodiments, it may be important that inner sheath 10 is fixed radially rather than axially. In those embodiments, tabs 10b may be used with slots or voids on the inner surface of the distal portion of connector 15 to hold inner sheath 10 in a radially-fixed position.

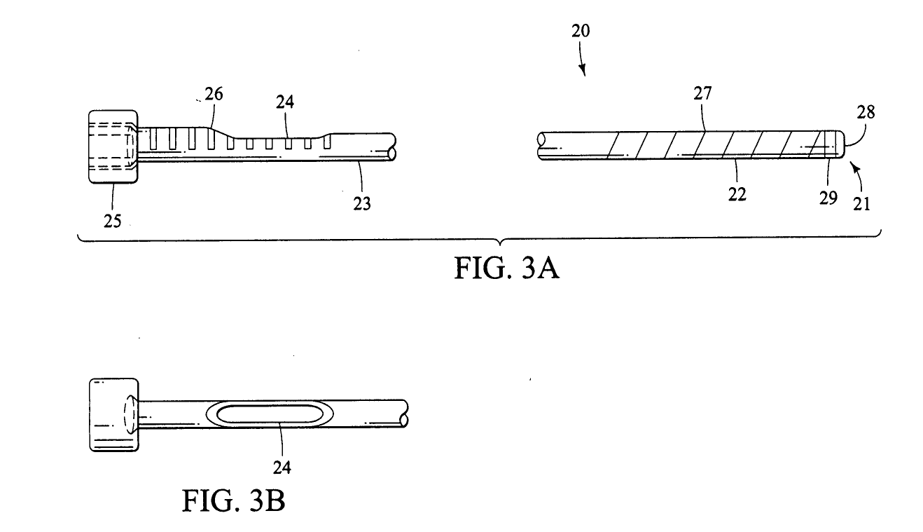

These components are used with a positioner as depicted in FIGS. 3a and 3b. Positioner 20 includes a distal portion 22, a proximal portion 23, and a hub 25. Hub 25 may be any convenient hub for interlocking with a mating connector on a cystoscope, as will be discussed below. Positioner 20 may also include a stress-relief or cut-out 24, allowing for easier use of the positioner inside the assembled delivery device. The cut-out portion 24 is preferably sufficient to allow easier use of the positioner in pushing the stent or other implant out of the outer sheath. In one embodiment, the cut-out is about 5-10 cm long, in an ovate shape, and is about 1-7 mm wide at the widest point. Cut-outs or strain reliefs with other dimensions may also be used.

Markings 26 on proximal portion 23, preferably at 1 cm intervals, aid a surgeon or clinician in placing a stent or other implant inside a person. Positioner 20 may also be made flexible by spiral cuts 27 in distal portion 22. Distal portion 22 may also have a slight taper or edge break 21, as shown on the distal end. The positioner is hollow, having a lumen 28 through the center, so that a cystoscope may be used on the inner portion of the delivery device.

The positioner is ideally used by visualization with the cystoscope mentioned above and the markings at 1-cm intervals on the proximal end of the positioner. In one embodiment, however, the positioner may have a radiopaque band 29 at its distal end for better visualization with a x-rays or a fluoroscope or other device using penetrating radiation. A radiopaque band is preferably a cylinder of stainless steel or other metal. In other embodiments, a radiopaque band may be placed onto the positioner, as disclosed in U.S. Pat. No. 5,300,048. The band may instead be echogenic, such that it is more easily seen with ultrasound techniques. In these embodiments, a series of radiation-reflecting features is machined into the surface of a preferably stainless steel band, such as hemispheres, cuts, grooves, and the like, as described in U.S. Pat. No. 6,527,752, assigned to the assignee of the present application, and incorporated by reference in its entirety.

As mentioned above, certain components of the delivery device are assembled in place and secured with respect to each other. For instance, inner sheath 10 locks to the inner surface of connector 15 and outer sheath 12 is fixedly mounted to an outer, threaded surface of connector 15. By tightening nut 18 on connector 15, positioner 20 may also be fixedly mounted to, or fixed in place with respect to, connector 15. When nut 18 is tightened, fingers 19 are forced radially inward, grasping positioner 20 and holding the positioner in place. In one embodiment, the components are designed so that when assembled, fingers 19 of connector 15 fit into the cut-out portion 24 of positioner 20.

By using hub 25, positioner 20 may also be fixedly mounted to a cystoscope, endoscope, or other device for visualizing inside a person's body, to assist in the placement of a stent or other device inside a patient. In this manner, the positioning device may be advanced through the body of the patient to the point where placement of the stent is desired, with assurance that all components of the implant delivery device are working together.

At some point during assembly of the positioning device, the stent or other medical device to be implanted is also assembled into the positioning device. This is typically done only after a surgeon evaluates the dimensions, usually length and diameter, of the stent or other device to be placed. Thus, a cystoscope, an endoscope, or other device typically is used to visualize the area of concern within the patient, and is then withdrawn for assembly into the positioning device.

At this point, the stent or other medical device may be chosen for a properly-fitting diameter, and the length may also be measured or estimated. The stent is then chosen and may be cut to length, before being placed inside the sheath or sheaths, and the positioner may be placed adjacent the stent. The several components are then locked in place as described above, with the stent, the positioner, the inner sheath and the outer sheath all fixed in place with respect to the connector. The assembly is then back-loaded onto a cystoscope or other device for implantation. The hub on the positioner locks the delivery device assembly in place with respect to the cystoscope, and the entire device is ready for placement. The cystoscope may also have an irrigation channel for visualization, as well as other features well known in the art.

It is important that the distal end of the implant delivery device be flexible, as flexible as the distal tip or end of a cystoscope. These scopes can typically bend 90° on a short radius, allowing for maximum visibility and a good field of view for the surgeon. In order for the implant delivery device to have this flexibility, the positioner, the outer sheath, and if necessary, the inner sheath, are designed and manufactured with this need in mind. The inner sheath, typically shorter than the outer sheath, is made of stiff, less pliable material, such as PTFE, so that the user is better able to manipulate the implant delivery device. The outer sheath, typically longer, and holding the stent, is made of softer, more compliant material, such as ePTFE (expanded PTFE). Other materials may be used.

The positioner is made of a relatively stiff material, so that it has good column strength when the surgeon wishes to move the positioner forward and place the stent or other device into a patient. The positioner is typically made of PENRT, and the hub may be acetal or other medically acceptable plastic, such as nylon or polycarbonate, or other material. The distal end of the positioner should be flexible, preferably as flexible as the cystoscope. A spiral cut may be placed on a distal portion of the positioner. A spiral cut may be made at an angle of 40 to 80 degrees to a longitudinal axis of the positioner, in which 90 degrees is perpendicular to the longitudinal axis. The spiral cut may have a pitch of about 6 mm, or the pitch may be from about 4 mm to about 12 mm. The pitch is the distance from cut to cut along a longitudinal axis of the positioner. The length of the spiral cut section may be from about 5 to about 15 cm long. Other pitches and lengths may be used, so long as the positioner has strength sufficient to push the stent out of the outer sheath and into the desired area in the patient (or to allow the sheath to retract), and flexibility sufficient to bend and flex with the cystoscope.

In practice, certain lengths and diameters are preferred. For instance, the inner sheath, made of a stiffer material, must have an inner diameter sufficient to pass the stent and a cystoscope, in some embodiments typically about 24 Fr. The inner sheath preferably also has an outer diameter of about 24.5 Fr, and is about 20 cm long. In one embodiment, the inner sheath is made from PTFE or other fluorocarbon compound, and has a wall thickness of about 0.002 inches to about 0.005 inches. The outer sheath, more flexible and pliable than the inner sheath, fits snugly over the inner sheath. Thus, the inner diameter of the outer sheath in these embodiments preferably is about 24.5 Fr. The outer diameter of the outer sheath is about 25 Fr, and thus the outer sheath also has a wall thickness of about 0.002 to about 0.005 inches. The outer sheath may be about 30-32 cm long, somewhat longer than the inner sheath.

The positioner must fit within the inner sheath and abut against the stent, so that the surgeon may use the positioner to push the stent or other implantable device into place. In this embodiment, the positioner thus may have an outer diameter no greater than 24 Fr. In addition, the positioner must allow a cystoscope or other device to pass through its central lumen, which in some embodiments is preferably about 16.5 Fr. The wall thickness of the positioner is thus about 0.050 inches, or from about 0.040 to about 0.060 inches. The positioner is preferably about 28-30 cm long, in addition to a hub, as described above, at its proximal end.

A cystoscope may be placed in the central lumen of the positioner, to guide the surgeon in the placement of the stent. Flexible cystoscopes are preferred, but rigid cystoscopes may also be used to deploy implants. In some embodiments, the cystoscope is preferably no more than about 24 Fr in diameter. In one embodiment, the cystoscope is preferably no more than about 16.3 Fr in diameter, and preferably possesses a highly flexible distal end. With this degree of flexibility, the surgeon can more easily visualize the area of interest where the stent or other implant is to be implanted, and can also use the cystoscope to help position the distal end of the outer sheath and the stent or implant devices itself. The entire length of the device thus includes the cystoscope, the positioner, the connector, the outer sheath and the stent or other device. In the case of an implant delivery device for a prostatic stent for a urethra of a male, the maximum length is about 38-40 cm, and the maximum width is about 26 Fr. Prostatic stents are from about 4 to about 10 cm in length, preferably from about 4.5 cm to about 9.5 cm. Other lengths may be used.

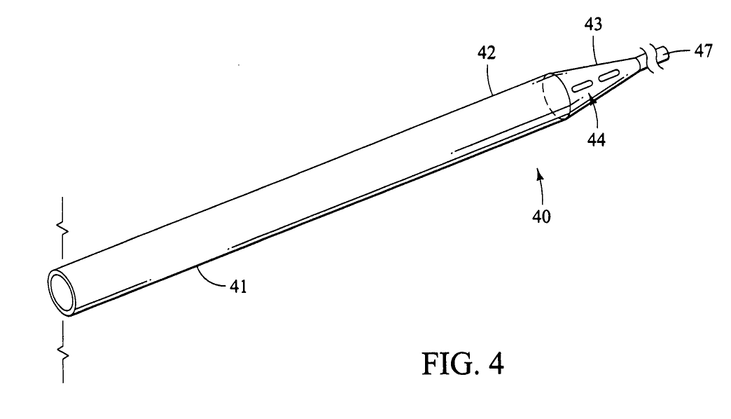

A prostatic stent may be implanted with the implant delivery device as described above. Other embodiments for components of the implant delivery device may also be used. For instance, some stents or devices to be implanted may tend to expand radially to an extent greater than prostatic stents. Thus, it may be useful to have an outer sheath that tends to compress the implant to at least a small extent before the implant is delivered to an area of interest in a patient's body. FIG. 4 depicts such an outer sheath 40. This embodiment of an outer sheath may be made from e-PTFE or other relatively pliable and flexible material. It may also be made from stiffer materials, such as PTFE, where a more rigid cystoscope is used. The sheath has a proximal portion 41 for mounting onto connector 15, and a distal portion 42. Distal portion 42 may also include a tapered end portion 43 with a diameter somewhat less than the implant or stent.

The end portion 43 may include one or more slits 44. A cystoscope 47 protrudes from end portion 43 so that a surgeon may direct the placement of the implant delivery device and the implant itself. When the desired area of placement is reached, the surgeon pushes the implant or stent in a distal direction, forcing the implant or stent into tapered end portion 43. Movement of the implant or stent into this narrower portion causes the slit or slits 44 to rupture, opening the distal end of the outer sheath, and allowing the implant or stent to be delivered. Alternatively, outer sheath 40 may be retracted, causing the sheath to rupture and delivering the implant.

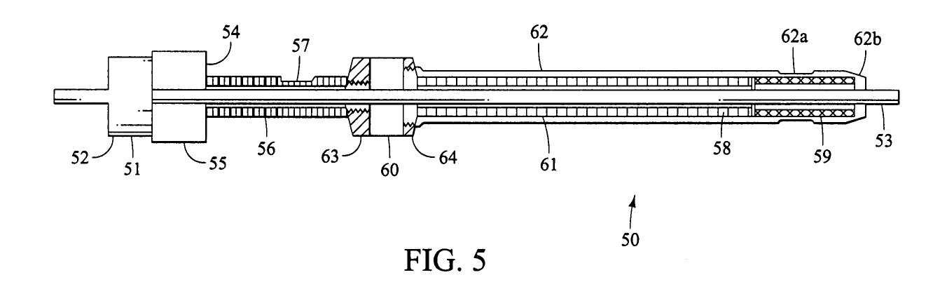

An assembly of the implant delivery device, along with a cystoscope and a stent, the cystoscope and stent not part of the implant delivery device, is depicted in FIG. 5. The assembly 50 makes use of a cystoscope 51, enabling a surgeon to use the implant delivery device and to implant a stent or other implant. Cystoscope 51 includes a connector 52 at the proximal end for connecting to the positioner 54 described above. The cystoscope extends through the assembly, to a very flexible distal end 53.

Positioner 54 includes a hub 55 for locking to cystoscope 51. Positioner 54 also includes a series of markings 56 at the proximal end, as discussed above, and a relief or cut-out portion 57. The distal end 58 of the positioner extends to the stent 59 or other implant. Assembly 50 also includes connector 60, to which are affixed an inner sheath 61 and an outer sheath 62. Inner sheath 61 is shorter than outer sheath 62, and extends only part way toward the distal end of the assembly. Outer sheath 62 extends distally to cover the implant device or stent 59, and may include slits 62a and a tapered distal end 62b. Varying the lengths and flexibility of inner sheath 61 and outer sheath 62 allows a user another way to control the flexibility and stiffness of the assembly. As described above with respect to FIG. 2, connector 60 includes nut 63 to compress and hold positioner 54 and nut 64 to affix outer sheath 62 to connector 60.

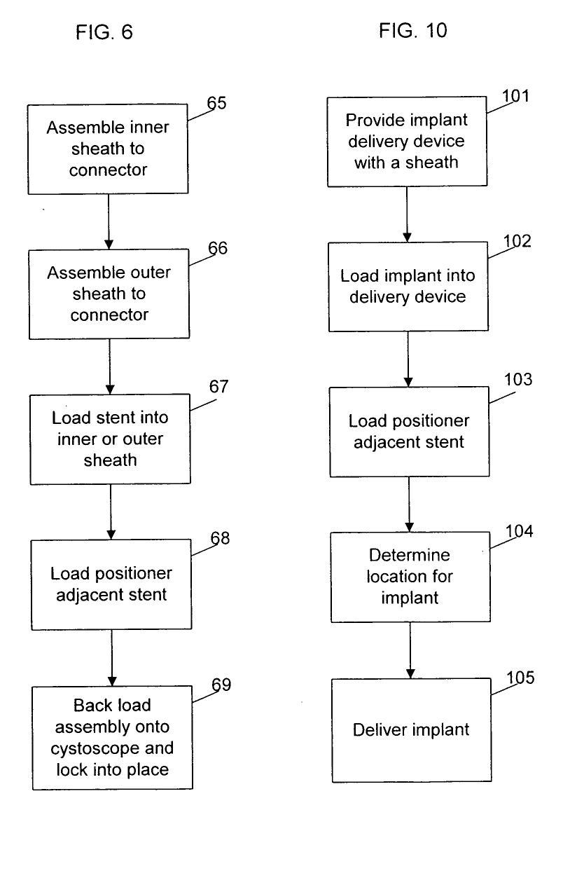

There are many uses for the implant delivery device. FIG. 6 is a flowchart for one method of using a device for implanting a stent or other prosthesis into the body. For any particular patient, the first step is to evaluate whether a stent, prosthesis, or other implant is indicated as medically necessary. Assuming that an implant is required, a surgeon must first estimate or measure the size of the implant. When these preliminary steps have been completed, the surgeon or a clinician selects an implant and an implant delivery device according to the limits of the device and the areas of the body the delivery device must traverse to reach the desired implant location.

To assemble an implant delivery device, an inner sheath is selected and assembled 65 to the connector, as outlined above, by an adhesive or a mechanical connection to the connector. An outer sheath is selected and is mounted 66 to the connector, preferably using a nut and threads. The outer sheath may be mounted by any other convenient technique. The stent is then loaded 67 into the assembly, through the connector and the inner sheath. The positioner may then be loaded 68. The positioner with connector and inner and outer sheaths and an implant is now back-loaded 69 (through the proximal end of the positioner) onto a cystoscope and locked in place. The delivery device is now ready to implant the stent.

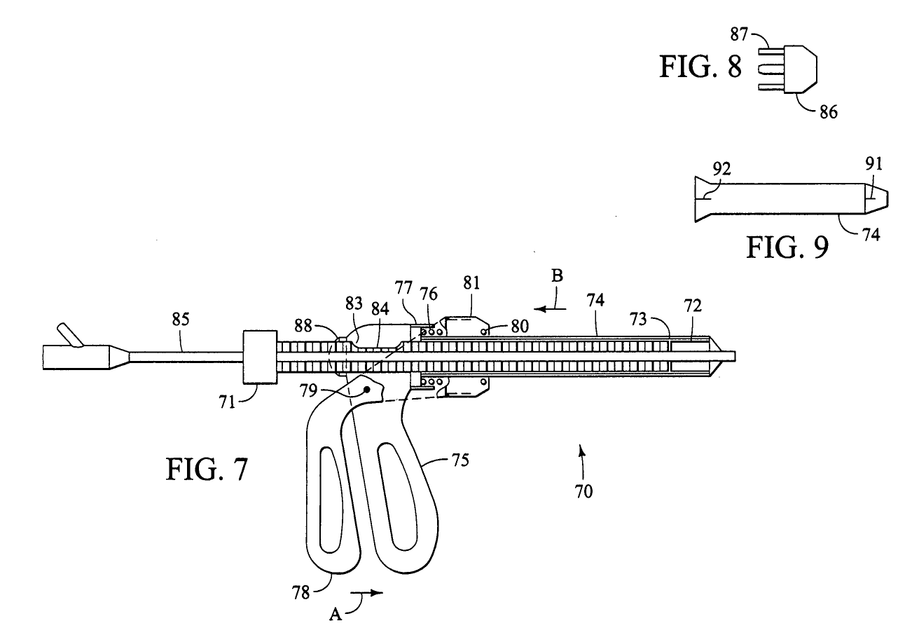

Other techniques include using an endoscope or other device for maneuvering and inspecting the body, which may also be used for delivering an implant as described herein. All manner of diagnostic and therapeutic devices are meant to be included. One such embodiment is depicted in FIG. 7, which depicts an assembly 70 including an implant delivery device with a handle for operating the device and a cystoscope 85 for visualizing the operating field. Assembly 70 includes a positioner 71 as described above, a stent 72, an inner sheath 73 and an outer sheath 74. Positioner 71 preferably includes a recess or relief 84 and a plurality of markings 83 preferably at about 1 cm intervals. These component parts are assembled as described above. Positioner 71 may include a compression fitting 88 to secure positioner 71 to fixed handle 75. Alternately, a locking feature or a secure attachment may be used to secure positioner 71 to fixed handle 75. The cystoscope could instead be locked in place by securing to the proximal hub of positioner 71.

The implant delivery device is operated by a handle including a fixed portion 75 and a movable portion or pivot handle 78, the two portions pivoting on pivot pin 79. Fixed portion 75 includes an upper mount 77 for a spring 76. Pivot handle 78 may threadably mount to a flare grip 81, which reacts against spring 76 to retract outer sheath 74 with every stroke of pivot handle 78. To use the assembly, a user strokes pivot handle 78, moving the lower portion distally, in the direction of arrow A, causing flare grip 81 to move proximally, in the direction of arrow B. When flare grip 81 moves proximally, a textured O-ring 80, or similar gripping device, causes outer sheath 74 to also move proximally. The amount of movement per stroke is determined by the travel allowed by flare grip 81, upper mount 77, and spring 76, which compresses with each stroke and then moves flare grip 81 and pivot handle 78 to their previous positions, ready for another stroke. When pivot handle 78 is released, a slight taper in flare grip 81 releases the grip of textured O-ring 80 on outer sheath 74. Spring 76 then urges flare grip 81 in a distal direction, the direction of arrow B, while outer sheath 74 remains in place. In one embodiment, the movement per stroke of the pivot handle is 2 cm, which also causes the outer sheath 74 to retract 2 cm. Other strokes may be used, e.g., 1 cm increments. Other handles may be used with the implant delivery device.

Other components may also be used with the handle depicted in FIG. 7. An alternate flare grip 86 is depicted in FIG. 8. With this grip, fingers 87 move radially inward when flare grip 86 is threaded onto handle 78. Fingers 87 may also be textured or roughened on their radially inward portions to better grasp outer sheath 74 and move the sheath in the proximal direction with each stroke of handle 78. For easier manipulation of the handle, outer sheath 74 in FIG. 9 may have two slits, slit 91 at the distal end as previously described, and slit 92 at the proximal end. When the sheath is retracted as described for FIG. 7, distal slit 91 enables easier deployment of stent 72. At least one proximal slit 92 enables easier manipulation of the handle, as the outer sheath proximal portion peels away in 1 cm or 2 cm increments, or other increments per stroke as desired.

There are many ways of practicing the invention. For instance, rather than using the implant delivery device as described above for delivering a prostatic stent for a urethra of a male, the device may be used to deliver a vascular stent, or repair tissue for a thorax, or any cavity or luminal support within the body. Engineered tissues, such as small intestine submucosa (SIS) derived from animal sources, may be used to repair a ruptured or broken vessel within a body, such as an aneurysm or trauma. Tissues for such repair are not limited to SIS, but the implant delivery device will work with a great variety of such tissue implants. The tissue implants are preferably tailored in their dimensions by the physician. These dimensions may include length and width, or they may be a diameter and length, as needed by the individual patient. All such devices and methods used for implanting these devices may be equivalent to the inventions described above and claimed in the claims attached hereto. While it is preferred to back-load the assembled delivery device onto a cystoscope or endoscope for delivery, the delivery device may be loaded by any convenient method. Positioning device embodiments may be used for positioning stents or implants a distance of from 5 mm to 45 cm within the body.

FIG. 10 is a flowchart for a method of delivering an implant. The method comprises a first step 101 of providing an implant delivery device further comprising a sheath preferably with a slit in the sheath. An implant is then loaded 102 into the delivery device, and a positioner with a flexible distal portion is loaded 103 adjacent the implant. The physician or surgeon then determines 104 a location for the implant using a therapeutic or diagnostic medical device. For purposes of this step, a therapeutic or diagnostic medical device may include an ultrasound machine or an x-ray machine, preferably determining the desired location using an echogenic or radiopaque tip on the positioner with a flexible distal portion. A therapeutic or diagnostic medical device may also include the cystoscope. The markings on the proximal portion of the positioner may also be used to determine the desired location of the implant, which is then delivered 105.

Any other undisclosed or incidental details of the construction or composition of the various elements of the disclosed embodiment of the present invention are not believed to be critical to the achievement of the advantages of the present invention, so long as the elements possess the attributes needed for them to perform as disclosed. The selection of these and other details of construction are believed to be well within the ability of even one of rudimentary skills in this area, in view of the present disclosure. For instance, the taper of the outer sheath need not be great, so long as the wider part is sufficient to hold the stent or other implant, so that the positioner can push the implant out of the sheath and into position in the body where the implant is desired. This taper may be from about 5 degrees to about 45 degrees. Other tapers may be used.

Illustrative embodiments of the present invention have been described in considerable detail for the purpose of disclosing a practical, operative structure whereby the invention may be practiced advantageously. The designs described herein are intended to be exemplary only. The novel characteristics of the invention may be incorporated in other structural forms without departing from the spirit and scope of the invention. The invention encompasses embodiments both comprising and consisting of the elements described herein with reference to the illustrative embodiments. Unless otherwise indicates, all ordinary words and terms used herein shall take their customary meaning as defined in the New Shorter Oxford English Dictionary, 1993 edition. All technical terms not defined herein shall take on their customary meaning as established by the appropriate technical discipline utilized by those normally skilled in that particular art area. All medical terms not otherwise defined shall take their meaning as defined by Stedman's Medical Dictionary, 27th edition. It is therefore intended that the foregoing detailed description be regarded as illustrative rather than limiting, and that it is desired to protect all variations and modifications within the valid scope of the present invention. It is intended that the scope and spirit of the invention be defined by the following claims, including all equivalents.

1. A positioning device, comprising:
an outer sheath;
an inner sheath within the outer sheath;
a positioner comprising a proximal portion and a flexible distal portion; and
a connector, wherein the outer sheath mounts to the connector and the positioner mounts to an inside of the connector.

2. The positioning device of claim 1, wherein the outer sheath is more conformable than the inner sheath.

3. The positioning device of claim 1, wherein the connector further comprises fingers for securing the positioner.

4. The positioning device of claim 1, wherein the positioner further comprises a stress relief portion and a hub for connecting to a diagnostic or therapeutic instrument.

5. The positioning device of claim 1, wherein the flexible distal portion comprises a spiral cut.

6. The positioning device of claim 1, wherein at least a portion of the outer sheath is tapered.

7. The positioning device of claim 1 wherein the outer sheath further comprises at least one slit.

8. The positioning device of claim 1, wherein the positioner further comprises at least two marks on the proximal portion.

9. The positioning device of claim 1, wherein the positioner further comprises a radiopaque or an echogenic portion.

10. The positioning device of claim 1, further comprising a handle.

11. The positioning device of claim 10, wherein the handle comprises a stationary portion and a pivoting portion joined at a pivot point.

12. The positioning device of claim 10, wherein the outer sheath has at least one slit.

13. A positioning device, comprising:
a tapered outer sheath;
an inner sheath within the outer sheath;
a positioner having a proximal end and a distal end; and
a connector, wherein the outer sheath mounts to an outside of the connector and the positioner mounts to an inside of the connector.

14. The device of claim 13, wherein the outer sheath further comprises at least one slit.

15. The device of claim 13, wherein the positioner further comprises at least two marks on the proximal portion.

16. The positioning device of claim 13, wherein the connector further comprises fingers for securing the positioner.

17. The positioning device of claim 13, wherein the positioner further comprises a stress relief portion and a locking hub.

18. The positioning device of claim 13, wherein the positioner further comprises a flexible distal portion.

19. The positioning device of claim 13, further comprising a handle.

20. The positioning device of claim 19, wherein the outer sheath further comprises at least one slit and the positioner further comprises a flexible distal portion.

21. A device for positioning a urethral stent, the device comprising:
a pliable tapered outer sheath further comprising at least one slit;
a stiff inner sheath within the outer sheath;
a positioner having a proximal end and a flexible distal end; and
a connector, wherein the outer sheath and inner sheath mount fixedly to the connector.

22. The device of claim 21, wherein a proximal portion of the positioner further comprises a stress-relief.

23. A method of making a positioning device, the method comprising:
assembling an inner sheath to a connector;
assembling an outer sheath to the connector; and
loading a positioner through the connector and the sheaths, the positioner further comprising a flexible distal portion.

24. The method of claim 23, wherein the inner sheath is assembled to the connector by an adhesive.

25. A method of delivering an implant, the method comprising:
providing an implant delivery device further comprising a sheath with at least one slit;
loading an implant into the delivery device;
loading a positioner with a flexible distal portion adjacent the implant;
determining a location for the implant with a therapeutic or diagnostic medical device; and
delivering the implant.

26. The method of claim 25, wherein the step of determining is accomplished with at least one of proximal markings on the positioner, a radiopaque band on the positioner, or an echogenic tip on the positioner.

27. The method of claim 25, wherein the sheath comprises at least one of an inner sheath and a tapered outer sheath, and wherein the implant is loaded into one of the inner sheath and the outer sheath.

28. The method of claim 25, wherein the step of delivering the implant is accomplished by urging the positioner and the implant in distal direction.

29. The method of claim 25, wherein the step of delivering the implant is accomplished by using a handle to retract the sheath.

30. The method of claim 25, wherein the medical diagnostic or therapeutic device is a cystoscope.
